(19) United States
(12) Patent Application Publication
(10) Pub. No.: US 2008/0114041 A1
(43) Pub. Date: May 15, 2008

(54) Benzothiazolesulfonamides

(75) Inventors: William Brown, Quebec (CA); Shawn Johnstone, Quebec (CA); Paul Jones, Quebec (CA); Denis Labrecque, Quebec (CA); Mickael Maudet, Reims Cedex (FR); Christopher Walpole, Quebec (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalij (SE)

(21) Appl. No.: 11/721,636

(22) PCT Filed: Dec. 19, 2005
(86) PCT No.: PCT/SE05/01965

§ 371 Date: Jun. 13, 2007

(30) Foreign Application Priority Data
Dec. 21, 2004 (SE) 0403118-3

(51) Int. Cl. A61K 31/428; C07D 417/02; C07D 277/82

(52) U.S. Cl. 514/367; 548/152; 548/161

(57) ABSTRACT

The present invention relates to new compounds of formula I,

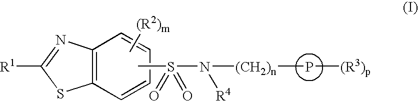

wherein R¹ to R⁴ are as defined as in formula I, or salts, solvates or solvated salts thereof, processes for their preparation and to new intermediates used in the preparation thereof, pharmaceutical formulations containing said compounds and to the use of said compounds in therapy.

### FIELD OF THE INVENTION

The present invention relates to new compounds, to pharmaceutical compositions containing said compounds and to the use of said compounds in therapy. The present invention further relates to processes for the preparation of said compounds and to new intermediates used in the preparation thereof.

### BACKGROUND OF THE INVENTION

Pain sensation in mammals is due to the activation of the peripheral terminals of a specialized population of sensory neurons known as nociceptors. Capsaicin, the active ingredient in hot peppers, produces sustained activation of nociceptors and also produces a dose-dependent pain sensation in humans. Cloning of the vanilloid receptor 1 (VR1 or TRPV1) demonstrated that VR1 is the molecular target for capsaicin and its analogues. (Caterina, M. J., et al., et. al. Nature (1997) v. 389 p 816-824). Functional studies using VR1 indicate that it is also activated by noxious heat, tissue acidification) and other inflammatory mediators (Tominaga, M., et. al. Neuron (1998) v. 21, p. 531-543). Expression of VR1 is also regulated after peripheral nerve damage of the type that leads to neuropathic pain. These properties of VR1 make it a highly relevant target for pain and for diseases involving inflammation. While agonists of the VR1 receptor can act as analgesics through nociceptor destruction, the use of agonists, such as capsaicin and its analogues, is limited due to their pungency, neurotoxicity and induction of hypothermia. Instead, agents that block the activity of VR1 should prove more useful. Antagonists would maintain the analgesic properties, but avoid pungency and neurotoxicity side effects.

Compounds with VR1 inhibitor activity are believed to be of potential use for the treatment and/or prophylaxis of disorders such as pain, especially that of inflammatory or traumatic origin such as arthritis, ischaemia, fibromyalgia, low back pain and post-operative pain (Walker et al., J Pharmacol Exp Ther. (2003) Jan; 304(1):56-62). In addition to this visceral pains such as chronic pelvic pain, cystitis, irritable bowel syndrome (IBS), pancreatitis and the like, as well as neuropathic pain such as sciatia, diabetic neuropathy, HIV neuropathy, multiple sclerosis, and the like (Walker et al ibid, J Pharmacol Exp Ther. (2003) Mar; 304(3):940-8), are potential pain states that could be treated with VR1 inhibiton. These compounds are also believed to be potentially useful for inflammatory disorders like asthma, cough, inflammatory bowel disease (IBD) (Hwang, et al., Curr Opin Pharmacol (2002) Jun; 2(3):235-42). Compounds with VR1 blocker activity are also useful for itch and skin diseases like psoriasis and for gastro-esophageal reflux disease (GERD), emesis, urinary incontinence and hyperactive bladder (Yiangou et al BJU Int (2001) Jun; 87(9):774-9, Szallasi, Am J Clin Pathol (2002) 118: 110-21). VR1 inhibitors are also of potential use for the treatment and/or prophylaxis of the effects of exposure to VR1 activators like capsaicin or tear gas, acids or heat (Szallasi ibid).

The role for VR1 antagonists in Inflammatory Bowel Diseases (IBD) is further supported by the finding that primary sensory neuron denervation by subcutaneous administration of capsaicin to neonatal rats, resulted in decreased levels of disease activity index (DAI), MPO and histological damage to the gut in DSS colitis model compared to control (N Kihara, et al., Gut, 2003. 52: p. 713-719). TRPV1 antagonists attenuate macroscopic symptoms in DSS colitis model in mice (E. S. KIMBALL, et al., Neurogastroenterol Motil, 2004. 16: p. 1-8).

The potential for a role for VR1 antagonists in Irritable Bowel Syndrome (IBS) has been described. Patients with faecal urgency and rectal hypersensitivity have increased levels of TRPV1 expression in nerve fibres in muscle, submucosal and mucosal layers. This also correlates with increase sensitivity to heat and distension (C L H Chan, et al., THE LANCET, 2003. 361(Feb 1): p. 385-91). Jejunal wide dynamic range (WDR) afferents show lower firing in response to pressure ex vivo in TRPV1-/-mice (Rong W, H. K., et al., J Physiol (Lond). 2004. 560: p. 867-881). The visceromotor responses to jejunal and colorectal distension in rat are affected by a TRPV1 antagonist using both ramp and phasic distensions (Winchester, EMG response to jejunal and colorectal distension in rat are affected by a TRPV1 antagonist in both ramp and phasic distensions. DDW abstract, 2004). Capsaicin applied to the ileum induce pain and mechanical hyperalgesia in human experimental model (Asbjørn Mohr Drewes, et al., Pain, 2003. 104: p. 333-341). A role in Gastroesophageal Reflux Disease (GERD) for VR1 antagonists has been mentioned in the literature. Patients with oesophagitis have increased levels of TRPV1 expression in peripheral nerves enervating the oesophageal epithelium (P. J. Matthews, et al., European J. of Gastroenterology & Hepatology, 2004. 16: p. 897-902). Even if the TRPV1 antagonist JYL1421 only has minor effects of acid-induced excitation of esophageal afferents, an antagonist with a different profile has yet to be evaluated. Since TRPV1 appears to play a role in mechanosensation, it is possible that antagonists may inhibit TLESRs, the main cause of gastroesophageal reflux.

A further portential use relates to the treatment of tolerance to VR1 activators. VR1 inhibitors may also be useful in the treatment of interstitial cystitis and pain related to interstitial cystitis.

### DETAILED DESCRIPTION OF THE INVENTION

The object of the present invention is to provide compounds exhibiting an inhibitory activity at the vanilloid receptor 1 (VR1).

The present invention provides a compound of formula I

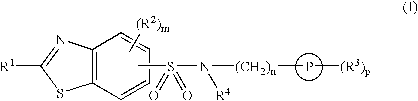

wherein:

ring P is C₆₋₁₀aryl, C₃₋₁₁cycloalkyl or C₅₋₁₀heteroaryl;

R¹ is H, C₁₋₄alkyl, hydroxyC₁₋₆alkyl, C₁₋₆alkylOC₀₋₆alkyl, COOC₀₋₆alkyl, NH₂, NHC₁₋₆alkyl, N(C₁₋₆alkyl)₂, NH(aryl) or N(aryl)₂;

R² is H, C₁₋₄alkyl, halo, hydroxyC₀₋₆alkyl or C₁₋₆alkylOC₀₋₆alkyl;

m is 0, 1, 2 or 3;

n is 0, 1, 2, 3, 4 or 5;

R³ is NO₂, NH₂C₀₋₆alkyl, halo, N(C₁₋₆alkyl)₂C₀₋₆alkyl, C₁₋₆alkyl, C₂₋₆alkenyl, C₂₋₆alkynyl, C₁₋₆haloalkyl, C₁₋₆haloalkylO, C₅₋₆arylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl, C₁₋₆alkylNC₀₋₆alkyl, (C₀₋₆alkyl)₂NC(O)C₀₋₆alkyl, (C₀₋₆alkyl)₂OC(O)C₀₋₆alkyl or (C₀₋₆alkyl)₂C(O)OC₀₋₆alkyl;

p is 1, 2, 3, 4 or 5; and

R⁴ is H, C₁₋₆alkyl, arylC₀₋₆alkyl, C₁₋₆alkylOC₀₋₆alkyl or N(C₁₋₆alkyl)₂C₀₋₆alkyl,

or salts, solvates or solvated salts thereof.

One embodiment of the invention relates to the compound of formula Ib, wherein R¹, R³, m, p and P are as described above and n is 0 and R² and R⁴ are H.

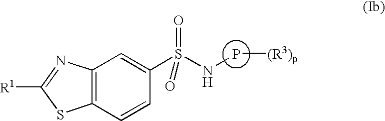

Another embodiment of the invention relates to the compound of formula Ic wherein R¹, R³, m, p and P are as described above and n is 1, 2, 3, 4 or 5 and R² and R⁴ are H.

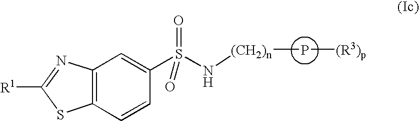

In a further embodiment of the invention P is phenyl.

In yet another embodiment of the invention R¹ is methyl or hydroxyC₁₋₃alkyl. In one embodiment R¹ is methyl, hydroxymethyl, hydroxyethyl or hydroxypropyl.

In another embodiment n is 0, 1 or 2.

In yet a further embodiment R³ is halo, C₁₋₃alkyl, C₁₋₃haloalkyl, C₅₋₆aryl, C₁₋₂alkylO or (C₀₋₆alkyl)₂NC(O)C₀₋₆alkyl.

In another embodiment R³ is phenyl, fluoromethyl, difluoromethyl or trifluoromethyl.

One embodiment of the invention relates to compounds selected from the group consisting of
2-(hydroxymethyl)-N-[4-(trifluoromethyl)phenyl]-1,3-benzothiazole-5-sulfonamide,
N-biphenyl-4-yl-2-(hydroxymethyl)-1,3-benzothiazole-5-sulfonamide,
2-(hydroxymethyl)-N-[3-(trifluoromethyl)phenyl]-1,3-benzothiazole-5-sulfonamide,
2-(hydroxymethyl)-N-[4-(trifluoromethyl)benzyl]-1,3-benzothiazole-5-sulfonamide,
2-(hydroxymethyl)-N-[3-(trifluoromethyl)benzyl]-1,3-benzothiazole-5-sulfonamide,
N-(4-isopropoxyphenyl)-2-methyl-1,3-benzothiazole-5-sulfonamide,
N-(4-tert-butylphenyl)-2-methyl-1,3-benzothiazole-5-sulfonamide,
2-methyl-N-[6-(trifluoromethyl)pyridin-3-yl]-1,3-benzothiazole-5-sulfonamide,
2-methyl-N-[3-(trifluoromethyl)phenyl]-1,3-benzothiazole-5-sulfonamide,
N-(4-bromophenyl)-2-methyl-1,3-benzothiazole-5-sulfonamide,
2-methyl-N-[2-(4-methylphenyl)ethyl]-1,3-benzothiazole-5-sulfonamide,
N-[2-(4-bromophenyl)ethyl]-2-methyl-1,3-benzothiazole-5-sulfonamide,
2-methyl-N-[2-(trifluoromethyl)benzyl]-1,3-benzothiazole-5-sulfonamide,
N-(4-bromo-3-fluorophenyl)-2-methyl-1,3-benzothiazole-5-sulfonamide,
2-methyl-N-[4-(trifluoromethyl)benzyl]-1,3-benzothiazole-5-sulfonamide,
N-[2-(4-tert-butylphenyl)ethyl]-2-methyl-1,3-benzothiazole-5-sulfonamide,
N-[2-(1H-indol-3-yl)ethyl]-2-methyl-1,3-benzothiazole-5-sulfonamide,
N-(4-iodobenzyl)-2-methyl-1,3-benzothiazole-5-sulfonamide,
N,N-diethyl-4-(2-{[(2-methyl-1,3-benzothiazol-5-yl)sulfonyl]amino}ethyl)benzamide,
2-methyl-N-[4-(trifluoromethoxy)benzyl]-1,3-benzothiazole-5-sulfonamide,
2-methyl-N-[(3-phenylisoxazol-5-yl)methyl]-1,3-benzothiazole-5-sulfonamide, and
2-methyl-N-[(2-phenyl-1,3-thiazol-4-yl)methyl]-1,3-benzothiazole-5-sulfonamide,
   or salts, solvates or solvated salts thereof.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, t-pentyl, neo-pentyl, n-hexyl, i-hexyl or t-hexyl. The term C₁₋₃ alkyl having 1 to 3 carbon atoms and may be methyl, ethyl, n-propyl or i-propyl.

The term ‘CO’ means “a bond” or “does not exist”. For example when R³ is C₀alkyl, R³ is a bond and “arylC₀alkyl” is equivalent with “aryl”, “C₂alkylOC₀alkyl” is equivalent with “C₂alkylO”.

In this specification, unless stated otherwise, the term “alkenyl” includes both straight and branched chain alkenyl groups. The term “C₂₋₆alkenyl” having 2 to 6 carbon atoms and one or two double bonds, may be, but is not limited to vinyl, allyl, propenyl, butenyl, crotyl, pentenyl, or hexenyl, and a butenyl group may for example be buten-2-yl, buten-3-yl or buten-4-yl.

In this specification, unless stated otherwise, the term “alkynyl” includes both straight and branched chain alkynyl groups. The term “C₂₋₆alkynyl” having 2 to 6 carbon atoms and one or two trippel bonds, may be, but is not limited to etynyl, propargyl, pentynyl or hexynyl and a butynyl group may for example be butyn-3-yl or butyn-4-yl.

In this specification, unless stated otherwise, the term “cycloalkyl” refers to an optionally substituted, saturated cyclic hydrocarbon ring system. The term “C₃₋₇cycloalkyl” may be cyclopropyl, cyclobutyl, cyclopentyl, cyclohexyl or cycloheptyl.

The term “heterocycloalkyl” denotes a 3- to 7-membered, non-aromatic, partially or completely saturated hydrocarbon group, which contains one ring and at least one heteroatom. Examples of said heterocycle include, but are not limited to pyrrolidinyl, pyrrolidonyl, piperidinyl, piperazinyl, morpholinyl, oxazolyl, 2-oxazolidonyl or tetrahydrofuranyl.

In this specification, unless stated otherwise, the term “aryl” refers to an optionally substituted monocyclic or bicyclic hydrocarbon unsaturated aromatic ring system. Examples of “aryl” may be, but are not limited to phenyl and naphthyl.

In this specification, unless stated otherwise, the term “heteroaryl” refers to an optionally substituted monocyclic or bicyclic ring system whereby at least one ring is aromatic independently from N, O or S. Examples of “heteroaryl” may be, but are not limited to pyridyl, pyrrolyl, furyl, thienyl, imidazolyl, oxazolyl, isoxazolyl, thiazolyl, pyrazolyl, benzofuryl, indolyl, isoindolyl, benzimidazolyl, pyridazinyl, pyrimidinyl, pyrazinyl, tetrazolyl, triazolyl or oxazolyl.

In this specification, unless stated otherwise, the terms “heteroarylalkyl” and “phenylalkyl” refer to a substituent that is attached via the alkyl group to an aryl or heteroaryl group.

In this specification, unless stated otherwise, the terms “halo” and “halogen” may be fluoro, iodo, chloro or bromo.

In this specification, unless stated otherwise, the term “haloalkyl” means an alkyl group as defined above, which is substituted with halo as defined above. The term “C₁₋₆haloalkyl” may include, but is not limited to fluoromethyl, difluoromethyl, trifluoromethyl, fluoroethyl, difluoroethyl or bromopropyl. The term “C₁₋₆haloalkylO” may include, but is not limited to fluoromethoxy, difluoromethoxy, trifluoromethoxy, fluoroethoxy or difluoroethoxy.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The present invention relates to the compounds of formula I as hereinbefore defined as well as to the salts, solvates or solvated salts thereof. Salts for use in pharmaceutical formulations will be pharmaceutically acceptable salts, but other salts may be useful in the production of the compounds of formula I.

A suitable pharmaceutically acceptable salt of the compounds of the invention is, for example, an acid-addition salt, for example a salt with an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.).

Some compounds of formula I may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of formula I.

Medical Use

Surprisingly, it has been found that the compounds according to the present invention are useful in therapy. The compounds of formula I, or salts, solvates or solvated salts thereof, as well as their corresponding active metabolites, exhibit a high degree of potency and selectivity for individual vanilloid receptor 1 (VR1) groups. Accordingly, the compounds of the present invention are expected to be useful in the treatment of conditions associated with excitatory activation of vanilloid receptor 1 (VR1).

The compounds may be used to produce an inhibitory effect of VR1 in mammals, including man.

VR1 are highly expressed the peripheral nervous system and in other tissues. Thus, it is expected that the compounds of the invention are well suited for the treatment of VR1 mediated disorders.

The compounds of formula I are expected to be suitable for the treatment of acute and chronic pain, acute and chronic neuropathic pain and acute and chronic inflammatory pain. Examples of such disorder may be selected from the group comprising arthritis, rheumatoid arthritis, spondylitis and gout, fibromyalgia, low back pain and sciatica, post-operative pain, cancer pain, migraine and tension headache, visceral pains like chronic pelvic pain, cystitis, including interstitial cystitis, pancreatitis, renal and biliary colic, menstruation associated pain, pain related to ischeamic and angina, neuropathic pain disorders such as diabetic neuropathy, HIV neuropathy, chemotherapy induced neuropathies, post-herpetic neuralgia, post traumatic neuralgia and complex regional syndrome as well as itch.

Further relevant disorders may be selected from the group comprising gastro-esophageal reflux disease (GERD), functional gastrointestinal disorders (FGD) such as irritable bowel syndrome (IBS), irritable bowel syndrome (IBS), and functional dyspepsia (FD).

Further examples of disorders are overactive bladder (“OAB”), a term for a syndrome that encompasses urge incontinence, urgency and frequency. Compounds of the invention may alleviate urinary incontinence (“UI”) the involuntary loss of urine that results from an inability of the bladder to retain urine as a consequence of either urge (urge incontinence), or physical or mental stress (stress incontinence).

Other relevant disorders may be psoriasis, and emesis.

Yet further relevant disorders are related to respiratory diseases and may be selected from the group comprising cough, asthma, chronic obstructive lung disease and emphysema, lung fibrosis and interstitial lung disease.

The VR1 inhibitor(s) for respiratory use, may be administrated by either an oral or inhaled route. The respiratory disease may be an acute and chronic illness and may be related to infection(s) and/or exposure to environmental pollution and/or irritants.

The compounds of formula I may also be used as antitoxin to treat (over-) exposure to VR1 activators like capsaicin, tear gas, acids or heat. Regarding heat, there is a potential use for VR1 antagonists in (sun-)burn induced pain, or inflammatory pain resulting from burn injuries.

The compounds may further be used for treatment of tolerance to VR1 activators.

One embodiment of the invention relates to the use of the compounds of formula I as hereinbefore defined, in therapy.

Another embodiment of the invention relates to the use of the compounds of formula I as hereinbefore defined, for treatment of VR1 mediated disorders.

A further embodiment of the invention relates to the use of the compounds of formula I as hereinbefore defined, for treatment of acute and chronic pain.

Yet another embodiment of the invention relates to the use of the compounds of formula I as hereinbefore defined, for treatment of acute and chronic neuropathic pain.

Yet a further embodiment of the invention relates to the use of the compounds of formula I as hereinbefore defined, for treatment of acute and chronic inflammatory pain.

One embodiment of the invention relates to the use of the compounds of formula I as hereinbefore defined, for treatment of arthritis, rheumatoid arthritis, spondylitis and gout, fibromyalgia, low back pain and sciatica, post-operative pain, cancer pain, migraine and tension headache, visceral pains like chronic pelvic pain, cystitis, including interstitial cystitis, pancreatitis, renal and biliary colic, menstruation associated pain, pain related to ischeamic and angina, neuropathic pain disorders such as diabetic neuropathy, HIV neuropathy, chemotherapy induced neuropathies, post-herpetic neuralgia, post traumatic neuralgia and complex regional syndrome as well as itch.

Another embodiment of the invention relates to the use of the compounds of formula I as hereinbefore defined, for treatment of gastro-esophageal reflux disease, functional gastrointestinal disorders, irritable bowel syndrome, irritable bowel syndrome and functional dyspepsia.

A further embodiment of the invention relates to the use of the compounds of formula I as hereinbefore defined, for treatment of overactive bladder.

Yet a further embodiment of the invention relates to the use of the compound of formula I as hereinbefore defined, for the treatment of respiratory diseases selected from the group comprising of cough, asthma, chronic obstructive lung disease and emphysema, lung fibrosis and interstitial lung disease.

One embodiment of the invention relates to the use of the compound of formula I as hereinbefore defined, in the manufacture of a medicament for treatment of VR1 mediated disorders and for treatment of acute and chronic pain, acute and chronic neuropathic pain and acute and chronic inflammatory pain, and respiratory diseases, and any other disorder mentioned above.

Another embodiment of the invention relates to a method of treatment of VR1 mediated disorders and acute and chronic pain, acute and chronic neuropathic pain and acute and chronic inflammatory pain, and respiratory diseases, and any other disorder mentioned above, comprising administrering to a mammal, including man in need of such treatment, a therapeutically effective amount of the compounds of formula I, as hereinbefore defined.

A further embodiment of the invention relates to a pharmaceutical composition comprising a compound of formula I as hereinbefore defined, for use in treatment of VR1 mediated disorders and for treatment of acute and chronic pain, acute and chronic neuropathic pain and acute and chronic inflammatory pain, and respiratory diseases, and any other disorder mentioned above.

In the context of the present specification, the term “therapy” and “treatment” includes prevention and prophylaxis, unless there are specific indications to the contrary. The terms “treat”, “therapeutic” and “therapeutically” should be construed accordingly.

In this specification, unless stated otherwise, the term “inhibitor” and “antagonist” mean a compound that by any means, partly or completely, blocks the transduction pathway leading to the production of a response by the ligand.

The term “disorder”, unless stated otherwise, means any condition and disease associated with vanilloid receptor activity.

Non-Medical Use

In addition to their use in therapeutic medicine, the compounds of the invention, or salts, solvates or solvated salts thereof, are also useful as pharmacological tools in the development and standardisation of in vitro and in vivo test systems for the evaluation of the effects of inhibitors of VR1 related activity in laboratory animals such as cats, dogs, rabbits, monkeys, rats and mice, as part of the search for new therapeutics agents.

Pharmaceutical Composition

According to one embodiment of the present invention there is provided a pharmaceutical composition comprising as active ingredient a therapeutically effective amount of the compound of formula I, or salts, solvates or solvated salts thereof, in association with one or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers. Suitable daily doses of the compounds of formula I in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

Examples of Pharmaceutical Composition

The following illustrate representative pharmaceutical dosage forms containing a compound of formula I, or salts, solvates or solvated salts thereof, (hereafter compound X) for preventive or therapeutic use in mammals:

| (a): Tablet | mg/tablet |
|---|---|
| Compound X | 100 |
| Lactose | 182.75 |
| Croscarmellose sodium | 12.0 |
| Maize starch paste (5% w/v paste) | 2.25 |
| Magnesium stearate | 3.0 |

| (b): Capsule | mg/capsule |
|---|---|
| Compound X | 10 |
| Lactose | 488.5 |
| Magnesium stearate | 1.5 |

| (c): Injection | (50 mg/ml) |
|---|---|
| Compound X | 5.0% w/v |
| 1M Sodium hydroxide solution | 15.0% v/v |
| 0.1M Hydrochloric acid | (to adjust pH to 7.6) |
| Polyethylene glycol 400 | 4.5% w/v |
| Water for injection | up to 100% |

The above compositions may be obtained by conventional procedures well known in the pharmaceutical art.

Methods of Preparation

General Methods of Preparation

One embodiment of the invention relates to a process for the preparation of the compound of formula I, wherein R¹ to R⁴, m, n and p, unless otherwise specified, are defined as in formula I, comprising;

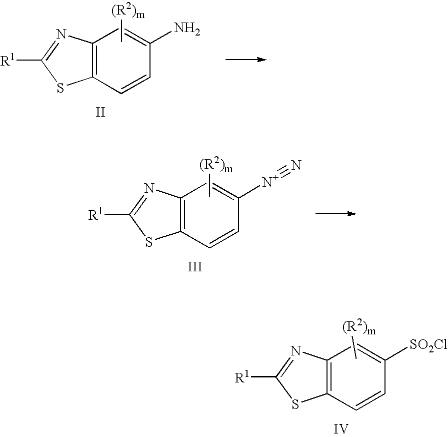

a) reaction of an aromatic amine of formula (II) with sodium nitrite in the presence of an acid such as hydrochloric acid, trifluoroacetic acid or acetic acid, to form a diazonium intermediate (III) which in turn may be reacted in-situ with sulphur dioxide or sodium sulfite in the presence of copper chloride to form an aromatic sulfonyl chloride (IV). This reaction may be performed in any manner known to the skilled person in the art. Suitable solvents to be used for this reaction may be water, acetone mixed with acids such as hydrochloric acid, sulphuric acid, acetic acid and TFA, or mixtures of the above. The temperature may be between 0 and 10° C. and the reaction time may be between 0.5 and 30 h.

Followed by

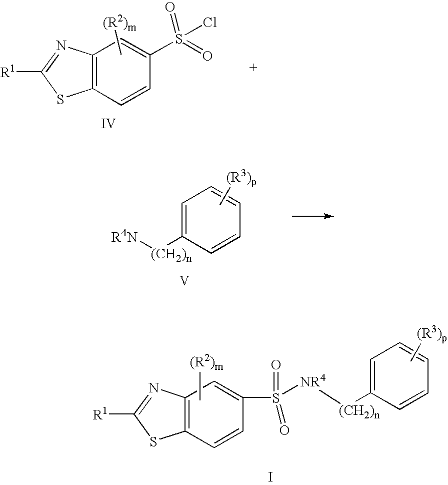

b) Reaction of an aromatic sulfonyl chloride (IV) with a properly substituted amine (V) in the presence of a base in a mixture of for example water and acetone. Suitable solvents to be used for this reaction may be tertiary amides such as dimethylformamide and dimethylacetamide, halogenated hydrocarbons such as chloroform, dichloromethane and dichloroethane or aromatic and heteroaromatic compounds such as benzene, toluene, xylene, pyridine and lutidine or ethers such as ethyl ether, tetrahydrofuran and dioxane, or any mixtures thereof.

Catalysts such as heteroaromatic bases like pyridine and lutidine or tertiary amines like triethylamine, N-methylmorpholine and ethyl diisopropylamine may be used as well.

The temperature may be between 10 and 60° C. and the reaction time may be between 3 and 30 h.

Another embodiment of the invention relates to a process for the preparation of the compound of formula I, wherein R¹ to R⁴, m, n and p, unless otherwise specified, are defined as in formula I, comprising;

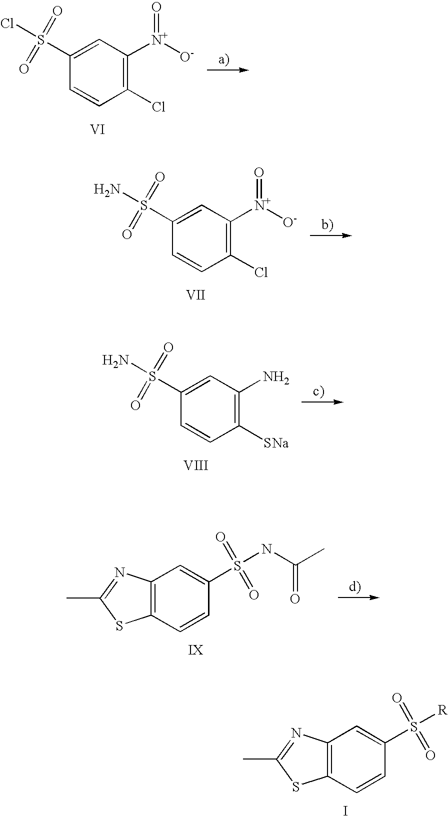

and wherein R is

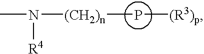

c) reaction of the sulfonylchloride VI with ammonia may be performed in suitable solvents like ethers or water, or any mixtures thereof, where ethers may be diethyl ether, dioxane, tetrahydrofurane and dimethylethylene glycol ether. Reaction of intermediate VII with sodium sulfide provides sulfide VIII, suitable solvents for this reaction may be water, acetonitrile, carbondisulfide, dimethylsulfoxide, or a mixture of thereof. Reaction of intermediate VIII to provide intermediate IX may be performed with acetic anhydride, acetic acid, or mixtures thereof, at about 100° C. followed by refluxing in acetic acid. Reaction of intermediate IX to provide the final compound I may be carried out in a two steps one pot sequence in which suitable solvents used in the first step may be POCl₃, dioxane, toluene. Suitable solvents to be used for the second step may be tertiary amides such as dimethylformamide and dimethylacetamide, halogenated hydrocarbons such as chloroform, dichloromethane and dichloroethane or aromatic and heteroaromatic compounds such as benzene, toluene, xylene, pyridine and lutidine or ethers such as ethyl ether, tetrahydrofuran and dioxane, or any mixtures thereof.

Catalist agent such as heteroaromatic bases like pyridine and lutidine or tertiary amines like triethylamine, N-methylmorpholine and ethyl diisopropylamine may be used as well. The temperature may be between 10 and 60° C. and the reaction time may be between 3 and 30 h.

Examples of specific conditions for the different process steps are

a) NH3, 1,4-Dioxane, rt b) Na2S.9H2O, H2O, 100° C. c) 1) Ac2O and AcOH, 100° C. 2) AcOH, reflux, d) 1) POCl3 reflux 2) CH2Cl2, DIPEA, Amine, rt.

The above-described processes may be performed in way known to the skilled person.

Intermediates

A further embodiment of the invention relates to compounds
N-[(2-methyl-1,3-benzothiazol-5-yl)sulfonyl]acetamide and allyl (5-amino-1,3-benzothiazol-2-yl)methyl carbonate,
   which may be used as intermediates in the preparation of compounds suited for the treatment of VR1 mediated disorders, especially for use as intermediates for the preparation of compounds of formula I.

### EXAMPLES

The invention will now be illustrated by the following Examples in which, generally:
(i) operations were carried out at ambient or room temperature, i.e. in the range 17 to 25° C. and under an atmosphere of an inert gas such as argon unless otherwise stated;
(ii) evaporations were carried out by rotary evaporation in vacuo and work-up procedures were carried out after removal of residual solids by filtration; column chromatography (by the flash procedure) was performed on Silicycle silica gel (grade 230-400 mesh, 60 Å, cat. Numb. R10030B) or obtained from Silicycle, Quebec, Canada or high pressure liquid chromatography (HPLC) was performed on C18 reverse phase silica, for example on a Phenomenex, Luna C-18 100 Å preparative reversed-phase column;

The ¹H NMR spectra were recorded on a Varian or Brucker at 400 or 600 MHz. The mass spectra were recorded utilising electrospray (LC-MS; LC:Waters 2790, column XTerra MS C₈ 2.5 μm 2.1×30 mm, buffer gradient H₂O+0.1% TFA:CH₃CN+0.04% TFA, MS: micromass ZMD//ammonium acetate buffer) ionisation techniques; yields, where present, are not necessarily the maximum attainable;
(vi) intermediates were not necessarily fully purified but their structures and purity were assessed by thin layer chromatographic, HPLC and/or NMR analysis
(vii) the following abbreviations have been used:
HPLC high performance liquid chromatography
LC liquid chromatography
MS mass spectometry
ret. time retention time
HCl hydrochloric acid
TFA trifluoroacetic acid
THF tetrahydrofuran
DIPEA N,N-diisopropylethylamine

General Procedure for the Preparation of acetyl sulphonamide intermediate IV used in examples 6 to 22.

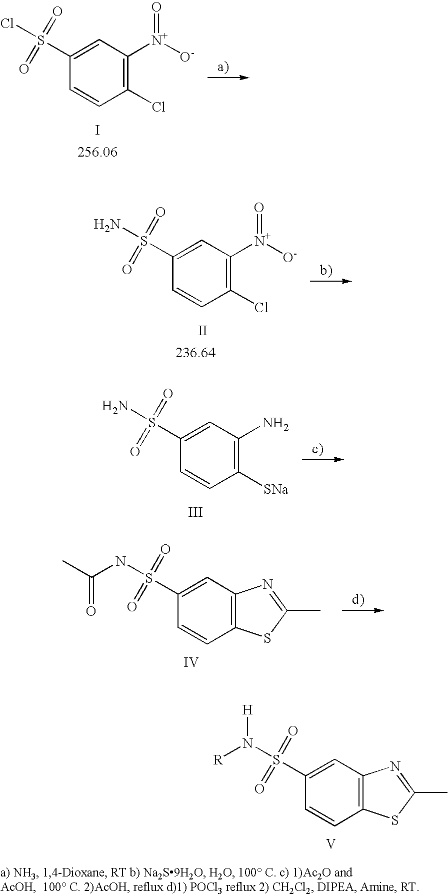

Intermediate 4-chloro-3-nitrobenzenesulfonamide II: To a solution of (7.5 g, 29.3 mmol) of 4-chloro-3-nitrobenzenesulfonyl chloride in dioxane 150 ml was bubbled NH₃ for one hour. The reaction was stirred until completion, then filtered, rinsed with dioxane and concentrated. The resulting solid was suspended in distilled water, filtered and dried. Yield 5.6 g, 23.7 mmol (80.7%). 1H NMR (400 MHz, DMSO-D6) δ ppm 7.75 (s, 2 H) 8.01 (d, J=8.40 Hz, 1 H) 8.07 (dd, J=8.40, 2.15 Hz, 1 H) 8.45 (d, J=2.15 Hz, 1 H).

Intermediate Sodium 2-amino-4-(aminosulfonyl)benzenethiolate III: To a suspension of 4-chloro-3-nitrobenzenesulfonamide (5.6 g, 23.7 mmol) in 100 ml of H₂O was added dropwise a solution of Na₂S.9H₂O over 10 min. The yellow suspension was heated at reflux for 2 hours then concentrated and used as such without isolation in the next step.

Intermediate N-[(2-methyl-1,3-benzothiazol-5-yl)sulfonyl]acetamide IV: Sodium 2-amino-4-(aminosulfonyl)benzenethiolate III was dissolved in 200 ml of acetic anhydride and heated for 1 hour at 100° C. To this was added 25 ml of acetic acid and the heating was continued for a further 1.5 hours. The reaction was then concentrated and taken into acetic acid and the reaction was heated at reflux until complete by LC-MS. The reaction was allowed to cool, filtered then rinsed with acetic acid followed by water. The resulting beige solid was dried under vacuum yielding 5.1 g, 79% of compound IV. 1H NMR (400 MHz, DMSO-D6) δ ppm 1.91 (s, 3 H) 2.85 (s, 3 H) 7.87 (dd, J=8.50, 1.86 Hz, 1 H) 8.30 (dd, J=8.50, 0.49 Hz, 1 H) 8.35 (d, J=1.37 Hz, 1 H) 12.19 (s, 1 H).

### Example 1

### 2-(hydroxymethyl)-N-[4-(trifluoromethyl)phenyl]-1,3-benzothiazole-5-sulfonamide

Allyl (5-amino-1,3-benzothiazol-2-yl)methyl carbonate (1.0 g, 3.78 mmol) is ground to a fine powder which is suspended in concentrated HCl (3.8 mL). The mixture is cooled to 5-10° C. and a solution of sodium nitrite (0.332 g, 4.81 mmol) in water (0.63 mL) is added dropwise. The mixture is stirred at 5-10° C. for 40 minutes and filtered under vacuum. While the diazotization reaction occurs sodium sulfite (1.192 g, 9.46 mmol) and copper sulfate (0.092 g, 0.575 mmol) are dissolved in concentrated HCl (8.8 mL) and water (2 mL). The mixture is cooled to 3-5° C. and the filtrate (from the diazotization reaction) is added followed by a solution of sodium nitrite (1.192 g, 9.46 mmol) in water (2 mL). The reaction is stirred at 3-5° C. for 1 hour and the precipitate is filtered, washed with water and dried under vacuum overnight. The sulfonyl chloride (0.727 g, 2.09 mmol) is dissolved in THF (6 mL). A saturated aqueous solution of sodium bicarbonate (1 mL) is added followed by 4-(trifluoromethyl)aniline (263 μL, 2.09 mmol). The reaction is stirred at room temperature for 1 hour. The aqueous phase is extracted twice with ethyl acetate. The combined organic phases are washed with brine, dried with anhydrous sodium sulfate, filtered and concentrated. The sulfonamide is dissolved in THF (25 mL) and 1M NaOH (25 mL) is added. The mixture is stirred at room temperature for 2 hours. Water is added and the aqueous phase is extracted twice with ethyl acetate. The combined organic phases are washed with water and brine, dried with sodium sulfate, filtered and concentrated. The crude is purified by Gilson reverse phase HPLC eluting with acetonitrile and water containing 0.1% TFA to yield the end product (41 mg, 5%). 1H NMR (600 MHz, MeOD) δ ppm 5.04 (s, 2 H) 7.38 (d, J=8.45 Hz, 2 H) 7.59 (d, J=8.45 Hz, 2 H) 7.91 (d, J=8.45 Hz, 1 H) 8.23 (d, J=8.45 Hz, 1 H) 8.42 (s, 1 H); MS [MH+] calc. 389.0. found 388.8.

### Example 2

### N-biphenyl-4-yl-2-(hydroxymethyl)-1,3-benzothiazole-5-sulfonamide

Allyl (5-amino-1,3-benzothiazol-2-yl)methyl carbonate (3.0 g, 11.35 mmol) is ground to a fine powder which is suspended in concentrated HCl (11.4 mL). The mixture is cooled to 5-10° C. and a solution of sodium nitrite (0.995 g, 14.42 mmol) in water (1.9 mL) is added dropwise. The mixture is stirred at 5-10° C. for 40 minutes and filtered under vacuum. While the diazotization reaction occurs sodium sulfite (3.577 g, 28.38 mmol) and copper sulfate (0.275 g, 1.73 mmol) are dissolved in concentrated HCl (26.4 mL) and water (6 mL). The mixture is cooled to 3-5° C. and the filtrate (from the diazotization reaction) is added followed by a solution of sodium nitrite (3.577 g, 28.32 mmol) in water (6 mL). The reaction is stirred at 3-5° C. for 1 hour and the precipitate is filtered, washed with water and dried under vacuum overnight. The sulfonyl chloride (0.400 g, 1.15 mmol) is dissolved in THF (4 mL). A saturated aqueous solution of sodium bicarbonate (1 mL) is added followed by 4-aminobiphenyl (0.195 g, 1. 15 mmol). The reaction is stirred at room temperature for 1 hour. The aqueous phase is extracted twice with ethyl acetate. The combined organic phases are washed with brine, dried with anhydrous sodium sulfate, filtered and concentrated. The sulfonamide is dissolved in THF (14 mL) and 1M NaOH (14 mL) is added. The mixture is stirred at room temperature for 2 hours. Water is added and the aqueous phase is extracted twice with ethyl acetate. The combined organic phases are washed with water and brine, dried with sodium sulfate, filtered and concentrated. The crude is purified by Gilson reverse phase HPLC eluting with acetonitrile and water containing 0.1% TFA to yield the end product (43 mg, 9%). 1H NMR (600 MHz, MeOD) δ ppm 4.93 (s, 2 H) 7.18 (d, J=8.70 Hz, 2 H) 7.27 (d, J=7.42 Hz, 1 H) 7.36 (t, J=7.68 Hz, 2 H) 7.46 (d, J=8.45 Hz, 2 H) 7.50 (d, J=7.68 Hz, 2 H) 7.79 (dd, J=8.45, 1.54 Hz, 1 H) 8.12 (d, J=8.71 Hz, 1 H) 8.28 (d, J=1.28 Hz, 1 H); MS [MH+] calc. 397.1. found 397.0.

### Example 3

### 2-(hydroxymethyl)-N-[3-(trifluoromethyl)phenyl]-1,3-benzothiazole-5-sulfonamide

Allyl (5-amino-1,3-benzothiazol-2-yl)methyl carbonate (3.0 g, 11.35 mmol) is ground to a fine powder which is suspended in concentrated HCl (11.4 mL). The mixture is cooled to 5-10° C. and a solution of sodium nitrite (0.995 g, 14.42 mmol) in water (1.9 mL) is added dropwise. The mixture is stirred at 5-10° C. for 40 minutes and filtered under vacuum.

While the diazotization reaction occurs sodium sulfite (3.577 g, 28.38 mmol) and copper sulfate (0.275 g, 1.73 mmol) are dissolved in concentrated HCl (26.4 mL) and water (6 mL). The mixture is cooled to 3-5° C. and the filtrate (from the diazotization reaction) is added followed by a solution of sodium nitrite (3.577 g, 28.32 mmol) in water (6 mL). The reaction is stirred at 3-5° C. for 1 hour and the precipitate is filtered, washed with water and dried under vacuum overnight. The sulfonyl chloride (0.400 g, 1.15 mmol) is dissolved in THF (4 mL). A saturated aqueous solution of sodium bicarbonate (1 mL) is added followed by 3-(trifluoromethyl)aniline (143 μL, 1.15 mmol). The reaction is stirred at room temperature for 2 hours. The aqueous phase is extracted twice with ethyl acetate. The combined organic phases are washed with brine, dried with anhydrous sodium sulfate, filtered and concentrated. The sulfonamide is dissolved in THF (14 mL) and 1M NaOH. (14 mL) is added. The mixture is stirred at room temperature for 2 hours. Water is added and the aqueous phase is extracted twice with ethyl acetate. The combined organic phases are washed with water and brine, dried with sodium sulfate, filtered and concentrated. The crude is purified by Gilson reverse phase HPLC eluting with acetonitrile and water containing 0.1% TFA to yield the end product (24mg, 5%). 1H NMR (600 MHz, MeOD) δ ppm 4.94 (s, 2 H) 7.30-7.35 (m, 2 H) 7.38 (d, J=7.94 Hz, 1 H) 7.40 (s, 1 H) 7.77 (dd, J=8.45, 1.54 Hz, 1 H) 8.13 (d, J=8.45 Hz, 1 H) 8.27 (d, J=1.28 Hz, 1 H); MS [MH+] calc. 389.0. found 388.8.

### Example 4

### 2-(hydroxymethyl)-N-[4-(trifluoromethyl)benzyl]-1,3-benzothiazole-5-sulfonamide

Allyl (5-amino-1,3-benzothiazol-2-yl)methyl carbonate (3.0 g, 11.35 mmol) is ground to a fine powder which is suspended in concentrated HCl (11.4 mL). The mixture is cooled to 5-10° C. and a solution of sodium nitrite (0.995 g, 14.42 mmol) in water (1.9 mL) is added dropwise. The mixture is stirred at 5-10° C. for 40 minutes and filtered under vacuum. While the diazotization reaction occurs sodium sulfite (3.577 g, 28.38 mmol) and copper sulfate (0.275 g, 1.73 mmol) are dissolved in concentrated HCl (26.4 mL) and water (6 mL). The mixture is cooled to 3-5° C. and the filtrate (from the diazotization reaction) is added followed by a solution of sodium nitrite (3.577 g, 28.32 mmol) in water (6 mL). The reaction is stirred at 3-5° C. for 1 hour and the precipitate is filtered, washed with water and dried under vacuum overnight. The sulfonyl chloride (0.400 g, 1.15 mmol) is dissolved in THF (4 mL). A saturated aqueous solution of sodium bicarbonate (1 mL) is added followed by 4-(trifluoromethyl)benzylamine (164 μL, 1.15 mmol). The reaction is stirred at room temperature for 2 hours. The aqueous phase is extracted twice with ethyl acetate. The combined organic phases are washed with brine, dried with anhydrous sodium sulfate, filtered and concentrated. The sulfonamide is dissolved in THF (14 mL) and 1M NaOH (14 mL) is added. The mixture is stirred at room temperature for 2 hours. Water is added and the aqueous phase is extracted twice with ethyl acetate. The combined organic phases are washed with water and brine, dried with sodium sulfate, filtered and concentrated. The crude is purified by Gilson reverse phase HPLC eluting with acetonitrile and water containing 0.1% TFA to yield the end product (44 mg, 9%). 1H NMR (600 MHz, MeOD) δ ppm 4.20 (s, 2 H) 4.97 (s, 2 H) 7.36 (d, J=8.19 Hz, 2 H) 7.44 (d, J=7.94 Hz, 2 H) 7.80 (dd, J=8.45, 1.54 Hz, 1 H) 8.12 (d, J=8.19 Hz, 1 H) 8.23 (s, 1 H); MS [MH+] calc. 403.0. found 402.7.

### Example 5

### 2-(hydroxymethyl)-N-[3-(trifluoromethyl)benzyl]-1,3-benzothiazole-5-sulfonamide

Allyl (5-amino-1,3-benzothiazol-2-yl)methyl carbonate (3.0 g, 11.35 mmol) is ground to a fine powder which is suspended in concentrated HCl (11.4 mL). The mixture is cooled to 5-10° C. and a solution of sodium nitrite (0.995 g, 14.42 mmol) in water (1.9 mL) is added dropwise. The mixture is stirred at 5-10° C. for 40 minutes and filtered under vacuum. While the diazotization reaction occurs sodium sulfite (3.577 g, 28.38 mmol) and copper sulfate (0.275 g, 1.73 mmol) are dissolved in concentrated HCl (26.4 mL) and water (6 mL). The mixture is cooled to 3-5° C. and the filtrate (from the diazotization reaction) is added followed by a solution of sodium nitrite (3.577 g, 28.32 mmol) in water (6 mL). The reaction is stirred at 3-5° C. for 1 hour and the precipitate is filtered, washed with water and dried under vacuum overnight. The sulfonyl chloride (0.400 g, 1.15 mmol) is dissolved in THF (4 mL). A saturated aqueous solution of sodium bicarbonate (1 mL) is added followed by 3-(trifluoromethyl)benzylamine (165 mL, 1.15 mmol). The reaction is stirred at room temperature for 2 hours. The aqueous phase is extracted twice with ethyl acetate. The combined organic phases are washed with brine, dried with anhydrous sodium sulfate, filtered and concentrated. The sulfonamide is dissolved in THF (14 mL) and 1M NaOH (14 mL) is added. The mixture is stirred at room temperature for 2 hours. Water is added and the aqueous phase is extracted twice with ethyl acetate. The combined organic phases are washed with water and brine, dried with sodium sulfate, filtered and concentrated. The crude is purified by Gilson reverse phase HPLC eluting with acetonitrile and water containing 0.1% TFA to yield the end product (8 mg; 2%). 1H NMR (600 MHz, MeOD) δ ppm 4.21 (s, 2 H) 4.97 (s, 2 H) 7.38 (d, J=7.68 Hz, 1 H) 7.42 (d, J=16.13 Hz, 2 H) 7.46 (d, J=7.42 Hz, 1 H) 7.79 (dd, J=8.45, 1.54 Hz, 1 H) 8.11 (d, J=8.45 Hz, 1 H) 8.27 (d, J=1.28 Hz, 1 H); MS [MH+] calc. 403.0. found 402.7.

### Example 6

### N-(4-isopropoxyphenyl)-2-methyl-1,3-benzothiazole-5-sulfonamide

200 mg of N-[(2-methyl-1,3-benzothiazol-5-yl)sulfonyl]acetamide was heated in POCl₃ at 110° C. overnight until complete conversion to the chloride. The solution was concentrated to dryness and placed under high vacuum. The brown oil was taken into 4 ml of dry CH₂Cl₂. To this was added 1.5 equivalents of 4-isopropoxyaniline and 3 equivalents of DIPEA. After stirring the reaction overnight, it was then concentrated, and taken into ethyl acetate and 1 N HCl. The aqueous phase is separated and the organic layer washed with 2 N NaHCO₃, then brine and dried over Na₂SO₄, filtered and concentrated. Purification was then done by silica gel chromatography with either ethyl acetate/heptane. 1H NMR (600 MHz, CDCl₃) δ ppm 1.29 (d, J=6.14 Hz, 6 H) 2.86 (s, 3 H) 4.41-4.48 (m, 1 H) 6.31 (s, 1 H) 6.72 (d, J=8.70 Hz, 2 H) 6.95 (d, J=8.96 Hz, 2 H) 7.64 (dd, J=8.45, 1.79 Hz, 1 H) 7.86 (d, J=8.19 Hz, 1 H) 8.31 (d, J=1.79 Hz, 1 H). MS [MH+] calc. 363.1. found 363.0.

### Example 7

### N-(4-tert-butylphenyl)-2-methyl-1,3-benzothiazole-5-sulfonamide

The procedure of example 6 was followed using 4-tert-butylaniline. 1H NMR (600 MHz, CDCl₃) δ ppm 1.24 (s, 9 H) 2.86 (s, 3 H) 6.74 (s, 1 H) 7.00 (d, J=8.19 Hz, 2 H) 7.23 (d, J=8.19 Hz, 2 H) 7.72 d, J=8.45 Hz, 1 H) 7.87 (d, J=8.19 Hz, 1 H) 8.39 (s, 1 H). MS [MH+] calc. 361.1. found 361.0.

### Example 8

### 2-methyl-N-[6-(trifluoromethyl)pyridin-3-yl]-1,3-benzothiazole-5-sulfonamide

The procedure of example 6 was followed using 3-amino-6-trifluromethylpyridine. 1H NMR (600 MHz, CDCl₃) δ ppm 2.92 (s, 3 H) 7.63 (dd, J=8.32, 2.18 Hz, 1 H) 7.73 (d, J=8.45 Hz, 1 H) 7.96 (dd, J=8.45, 1.79 Hz, 1 H) 8.05 (d, J=8.45 Hz, 1 H) 8.36 (d, J=2.30 Hz, 1 H) 8.45 (d, J=1.79 Hz, 1 H). MS [MH+] calc. 374.0. found 373.7

### Example 9

### 2-methyl-N-[3-(trifluoromethyl)phenyl]-1,3-benzothiazole-5-sulfonamide

The procedure of example 6 was followed using 3-trifluromethylaniline. 1H NMR (600 MHz, CDCl₃) δ ppm 2.87 (s, 3 H) 6.77 (s, 1 H) 7.29-7.34 (m, 2 H) 7.35-7.39 (m, 2 H) 7.73 (dd, J=8.45, 1.79 Hz, 1 H) 7.90 (d, J=8.19 Hz, 1 H) 8.38 (d, J=1.79 Hz, 1 H). MS [MH+] calc. 373.0. found 372.8.

### Example 10

### N-(4-bromophenyl)-2-methyl-1,3-benzothiazole-5-sulfonamide

The procedure of example 6 was followed using 4-bromoaniline. 1H NMR (600 MHz, DMSO-D6) δ ppm 2.83 (s, 3 H) 7.06 (d, J=8.96 Hz, 2 H) 7.41 (d, J=8.96 Hz, 2 H) 7.72 (d, J=8.45 Hz, 1 H) 8.21 (s, 1 H) 8.24 (d, J=8.45 Hz, 1 H) 10.56 (s, 1 H). MS [MH+] calc. 383.0. found 382.7.

### Example 11

### 2-methyl-N-[2-(4-methylphenyl)ethyl]-1,3-benzothiazole-5-sulfonamide

The procedure of example 6 was followed using 2-(p-tolyl)ethylamine. 1H NMR (600 MHz, DMSO-D6) δ ppm 2.21 (s, 3 H) 2.60 (t, J=7.42 Hz, 2 H) 2.84 (s, 3 H) 2.90-2.98 (m, 2 H) 6.97-7.03 (m, 4 H) 7.74 (dd, J=8.45, 1.54 Hz, 1 H) 7.79 (t, J=5.76 Hz, 1 H) 8.20 (d, J=1.54 Hz, 1 H) 8.24 (d, J=8.45 Hz, 1 H) MS [MH+] calc. 347.1. found 347.0.

### Example 12

### N-[2-(4-bromophenyl)ethyl]-2-methyl-1,3-benzothiazole-5-sulfonamide

The procedure of example 6 was followed using 4-bromophenethylamine. 1H NMR (600 MHz, DMSO-D6) δ ppm 2.64 (t, J=7.17 Hz, 2 H) 2.84 (s, 3 H) 2.95-3.00 (m, 2 H) 7.09 (d, J=8.45 Hz, 2 H) 7.37 (d, J=8.45 Hz, 2 H) 7.72 (dd, J=8.45, 1.79 Hz, 1 H) 7.80 (t, J=5.63 Hz, 1 H) 8.19 (d, J=1.79 Hz, 1 H) 8.23 (d, J=8.45 Hz, 1 H). MS [MH+] calc. 410.9. found 410.7.

### Example 13

### 2-methyl-N-[2-(trifluoromethyl)benzyl]-1,3-benzothiazole-5-sulfonamide

The procedure of example 6 was followed using 2-trifluoromethylbenzylamine. 1H NMR (400 MHz, DMSO-D6) δ ppm 2.85 (s, 3 H) 4.16 (d, J=6.05 Hz, 2 H) 7.42 (t, J=7.42 Hz, 1 H) 7.55-7.66 (m, 3 H) 7.77 (dd, J=8.40, 1.95 Hz, 1 H) 8.24 (d, J=1.76 Hz, 1 H) 8.26 (dd, J=8.40, 0.59 Hz, 1 H) 8.44 (t, J=6.15 Hz, 1 H). MS [MH+] calc. 387.0. found 386.7.

### Example 14

### N-(4-bromo-3-fluorophenyl)-2-methyl-1,3-benzothiazole-5-sulfonamide

The procedure of example 6 was followed using 4-bromo-3-fluoroaniline. 1H NMR (600 MHz, DMSO-D6) δ ppm 2.82 (s, 3 H) 6.90 (dd, J=8.70, 2.30 Hz, 1 H) 7.07 (dd, J=10.50, 2.30 Hz, 1 H) 7.54 (t, J=8.32 Hz, 1 H) 7.75 (dd, J=8.58, 1.66 Hz, 1 H) 8.25 (s, 1 H) 8.26 (d, J=5.89 Hz, 1 H) 10.85 (s, 1 H). MS [MH+] calc. 400.9. found 400.8.

### Example 15

### 2-methyl-N-[4-(trifluoromethyl)benzyl]-1,3-benzothiazole-5-sulfonamide

The procedure of example 6 was followed using 4-trifluoromethylbenzylamine. 1H NMR (600 MHz, DMSO-D6) δ ppm 2.84 (s, 3 H) 4.10-4.17 (m, 2 H) 7.43 (d, J=8.19 Hz, 1 H) 7.56 (d, J=8.19 Hz, 2 H) 7.76 (d, J=8.19 Hz, 2 H) 8.17 (s, 1 H) 8.23 (d, J=8.45 Hz, 1 H) 8.43 (t, J=6.53 Hz, 1 H). MS [MH+] calc. 387.0. found 386.8.

### Example 16

### N-[2-(4-tert-butylphenyl)ethyl]-2-methyl-1,3-benzothiazole-5-sulfonamide

The procedure of example 6 was followed using 2-(4-tert-butylphenyl)ethylamine. 1H NMR (400 MHz, DMSO-D6) δ ppm 1.22 (s, 9 H) 2.55-2.67 (m, 2 H) 2.84 (s, 3 H) 2.88-3.00 (m, 2 H) 7.02-7.07 (m, 2 H) 7.21-7.26 (m, 2 H) 7.76 (dd, J=8.40, 1.76 Hz, 1 H) 7.80 (t, J=5.86 Hz, 1 H) 8.22-8.27 (m, 2 H). MS [MH+] calc. 389.1. found 389.0.

### Example 17

### N-[2-(1H-indol-3-yl)ethyl]-2-methyl-1,3-benzothiazole-5-sulfonamide

The procedure of example 6 was followed using tryptamine. 1H NMR (400 MHz, DMSO-D6) δ ppm 2.76 (t, J=7.62 Hz, 2 H) 2.82-2.86 (m, 3 H) 2.97-3.05 (m, 2 H) 6.90 (t, 1 H) 6.98-7.04 (m, 1 H) 7.09 (d, J=2.15 Hz, 1 H) 7.30 (dd, J=16.01, 7.81 Hz, 2 H) 7.77 (dd, J=8.30, 1.86 Hz, 1 H) 7.86 (t, J=5.76 Hz, 1 H) 8.21-8.27 (m, 2 H) 10.79 (s, 1 H). MS [MH+] calc. 372.1. found 372.0.

### Example 18

### N-(4-iodobenzyl)-2-methyl-1,3-benzothiazole-5-sulfonamide

The procedure of example 6 was followed using 4-iodobenzylamine. 1H NMR (400 MHz, DMSO-D6) δ ppm 2.85 (s, 3 H) 3.96 (d, J=6.45 Hz, 2 H) 6.99 (d, J=8.59 Hz, 2 H) 7.53 (d, J=8.40 Hz, 2 H) 7.73 (dd, J=8.40, 1.95 Hz, 1 H) 8.14 (d, J=1.37 Hz, 1 H) 8.22 (dd, J=8.40, 0.59 Hz, 1 H) 8.29 (t, J=6.35 Hz, 1 H). MS [MH+] calc. 444.9. found 444.7.

### Example 19

### N,N-diethyl-4-(2-{[(2-methyl-1,3-benzothiazol-5-yl)sulfonyl]amino}ethyl)benzamide

The procedure of example 6 was followed using 4-(2-amino-ethyl)-N,N-diethylbenzamide. 1H NMR (400 MHz, DMSO-D6) δ ppm 0.98-1.14 (m, 6 H) 2.70 (t, J=7.32 Hz, 2 H) 2.84 (s, 3 H) 2.95-3.03 (m, 2 H) 3.15 (s, 2 H) 3.39 (s, 2 H) 7.15-7.23 (m, 4 H) 7.73-7.77 (m, 1 H) 7.84 (t, J=5.76 Hz, 1 H) 8.24 (s, 1 H) 8.25 (d, J=6.64 Hz, 1 H). MS [MH+] calc. 432.1. found 432.0.

### Example 20

### 2-methyl-N-[4-(trifluoromethoxy)benzyl]-1,3-benzothiazole-5-sulfonamide

The procedure of example 6 was followed using 4-trifluoromethoxybenzylamine. 1H NMR (400 MHz, DMSO-D6) δ ppm 2.84 (s, 3 H) 4.04 (d, J=6.25 Hz, 2 H) 7.20 (d, J=8.20 Hz, 2 H) 7.33 (d, J=8.79 Hz, 2 H) 7.74 (dd, J=8.40, 1.76 Hz, 1 H) 8.20 (d, J=1.76 Hz, 1 H) 8.22 (d, J=8.40 Hz, 1 H) 8.34 (t, J=6.45 Hz, 1 H). MS [MH+] calc. 403.0. found 402.7.

### Example 21

### 2-methyl-N-[(3-phenylisoxazol-5-yl)methyl]-1,3-benzothiazole-5-sulfonamide

The procedure of example 6 was followed using (3-phenyl-5-isoxazolyl)methanamine. 1H NMR (400 MHz, DMSO-D6) δ ppm 2.78 (s, 3 H) 4.28 (d, J=6.05 Hz, 2 H) 6.62 (s, 1 H) 7.41-7.48 (m, 3 H) 7.57-7.62 (m, 2 H) 7.75 (dd, J=8.40, 1.76 Hz, 1 H) 8.20 (d, J=8.40 Hz, 1 H) 8.22 (d, J=1.56 Hz, 1 H) 8.60 (t, J=6.25 Hz, 1 H). MS [MH+] calc. 386.1. found 385.8.

### Example 22

### 2-methyl-N-[(2phenyl-1,3-thiazol-4-yl)methyl]-1,3-benzothiazole-5-sulfonamide

The procedure of example 6 was followed using (2-phenyl-1,3-thiazol-4-yl)methylamine. 1H NMR (400 MHz, DMSO-D6)δ ppm 2.78 (s, 3 H) 4.19 (d, J=6.05 Hz, 2 H) 7.37-7.45 (m, 4H) 7.63-7.72 (m, 3 H) 8.11 (d, J=8.40 Hz, 1 H) 8.20 (d, J=1.56 Hz, 1 H) 8.38 (t, J=6.15 Hz, 1 H). MS [MH+] calc. 402.0. found 401.7.

Pharmacology

1. hVR1 FLIPR (Fluorometric Image Plate Reader) Screening Assay

Transfected CHO cells, stably expessing hVR1 (15,000 cells/well) are seeded in 50 ul media in a black clear bottom 384 plate (Greiner) and grown in a humidified incubator (37° C., 2% CO₂), 24-30 hours prior to experiment.

Subsequently, the media is removed from the cell plate by inversion and 2 μM Fluo-4 is added using a multidrop (Labsystems). Following the 40 minutes dye incubation in the dark at 37° C. and 2% CO₂, the extracellular dye present is washed away using an EMBLA (Scatron), leaving the cells in 40 ul of assay buffer (1×HBSS, 10 mM D-Glucose, 1 mM CaCl₂, 10 mM HEPES, 10×7.5% NaHCO₃ and 2.5 mM Probenecid).

FLIPR Assay—IC₅₀ Determination Protocol

For IC₅₀ determinations the fluorescence is read using FLIPR filter 1 (em 520-545 nM). A cellular baseline recording is taken for 30 seconds, followed by a 20 μl addition of 10, titrated half-log concentrations of the test compound, yielding cellular concentration ranging from 3 μM to 0.1 nM. Data is collected every 2 seconds for a further 5 minutes prior to the addition of a VR1 agonist solution: either 50 nM solution of capsaicin or MES (2-[N-morpholino]ethanesulfonic acid) buffer (pH 5.2), by the FLIPR pipettor. The FLIPR continues to collect data for a further 4 minutes. Compounds having antagonistic properties against the hVR1 will inhibit the increase in intracellular calcium in response to the capsaicin addition. This consequently leading to a reduction in fluorescence signal and providing a reduced fluorescence reading, compared with no compound, buffer controls. Data is exported by the FLIPR program as a sum of fluorescence calculated under the curve upon the addition of capsaicin. Maximum inhibition, Hill slope and IC₅₀ data for each compound are generated.

List of Abbreviations
VR1 vanilloid receptor 1
IBS irritable bowel syndrome
IBD inflammatory bowel disease
GERD gastro-esophageal reflux disease
HEPES 4-(2-Hydroxyethyl)piperazine-1-ethanesulfonic acid
EGTA Ethylene glycol-bis(2-aminoethylether)-N,N,N′,N′-tetraacetic acid
EMBLA Skatron, Plate Cell Washer, from Molecular Devices company
HBSS Hank's Balanced Salt Solution
MES (2-[N-Morphholino]ethanesulfonic acid) Hydrate, Sigma cat# M-5287
NUT Nutrient mixture F-12, medium for culturing cells
MEM Minimal Eagle Medium

Results

Typical IC₅₀ values as measured in the assays described above are 10 μM or less. In one aspect of the invention the IC₅₀ is below 10 μM.

TABLE 1
Specimen results from the hVR1 FLIPR.

| Compound No. | IC₅₀ nM (agonist) |
|---|---|
| 5 | 2870 |

1. A compound of formula I 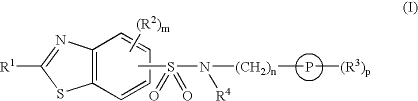
wherein:
ring P is C₆₋₁₀aryl, C₃₋₁₁cycloalkyl or C₅₋₁₀heteroaryl;
R¹ is H, C₁₋₄alkyl, hydroxyC₁₋₆alkyl, C₁₋₆alkylOC₀₋₆alkyl, COOC₀₋₆alkyl, NH₂, NHC₁₋₆alkyl, N(C₁₋₆alkyl)₂, NH(aryl) or N(aryl)₂;
R² is H, C₁₋₄alkyl, halo, hydroxyC₀₋₆alkyl or C₁₋₆alkylOC₀₋₆alkyl;
m is 0, 1, 2 or 3;
n is 0, 1, 2, 3, 4 or 5;
R³ is NO₂, NH₂C₀₋₆alkyl, halo, N(C₁₋₆alkyl)₂C₀₋₆alkyl, C₁₋₆alkyl, C₂₋₆alkenyl, C₂₋₆alkynyl, C₁₋₆haloalkyl, C₁₋₆haloalkylO, C₅₋₆arylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl, C₁₋₆alkylNC₀₋₆alkyl, (C₀₋₆alkyl)₂NC(O)C₀₋₆alkyl, (C₀₋₆alkyl)₂OC(O)C₀₋₆alkyl or (C₀₋₆alkyl)₂C(O)OC₀₋₆alkyl;
p is 1, 2, 3, 4 or 5; and
R⁴ is H, C₁₋₆alkyl, arylC₀₋₆alkyl, C₁₋₆alkylOC₀₋₆alkyl or N(C₁₋₆alkyl)₂C₀₋₆alkyl,
or salts, solvates or solvated salts thereof.

2. A compound of formula Ib wherein wherein R¹, R³, m, p and P are as defined as in claim 1, and n is 0 and R² and R⁴ are H.

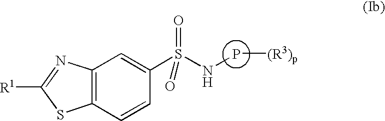

3. A compound of formula Ic, wherein R¹, R³, p, m and P are as defined as in claim 1, and n is 1, 2, 3, 4 or 5 and R² and R⁴ are H.

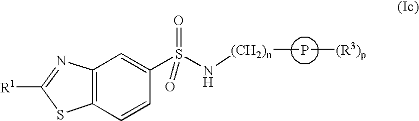

4. A compound according to claim 1 wherein ring P is phenyl.

5. A compound according to claim 1 wherein R¹ is methyl or hydroxyC₁₋₃alkyl.

6. A compound according to claim 1 wherein R³ is phenyl, fluoromethyl, difluoromethyl or trifluoromethyl.

7. A compound selected from the group consisting of:
2-(hydroxymethyl)-N-[4-(trifluoromethyl)phenyl]-1,3-benzothiazole-5-sulfonamide,
N-biphenyl-4-yl-2-(hydroxymethyl)-1,3-benzothiazole-5-sulfonamide,
2-(hydroxymethyl)-N-[3-(trifluoromethyl)phenyl]-1,3-benzothiazole-5-sulfonamide,
2-(hydroxymethyl)-N-[4-(trifluoromethyl)benzyl]-1,3-benzothiazole-5-sulfonamide,
2-(hydroxymethyl)-N-[3-(trifluoromethyl)benzyl]-1,3-benzothiazole-5-sulfonamide,
N-(4-isopropoxyphenyl)-2-methyl-1,3-benzothiazole-5-sulfonamide,
N-(4-tert-butylphenyl)-2-methyl-1,3-benzothiazole-5-sulfonamide,
2-methyl-N-[6-(trifluoromethyl)pyridin-3-yl]-1,3-benzothiazole-5-sulfonamide,
2-methyl-N-[3-(trifluoromethyl)phenyl]-1,3-benzothiazole-5-sulfonamide,
N-(4-bromophenyl)-2-methyl-1,3-benzothiazole-5-sulfonamide,
2-methyl-N-[2-(4-methylphenyl)ethyl]-1,3-benzothiazole-5-sulfonamide,
N-[2-(4-bromophenyl)ethyl]-2-methyl-1,3-benzothiazole-5-sulfonamide,
2-methyl-N-[2-(trifluoromethyl)benzyl]-1,3-benzothiazole-5-sulfonamide,
N-(4-bromo-3-fluorophenyl)-2-methyl-1,3-benzothiazole-5-sulfonamide,
2-methyl-N-[4-(trifluoromethyl)benzyl]-1,3-benzothiazole-5-sulfonamide,
N-[2-(4-tert-butylphenyl)ethyl]-2-methyl-1,3-benzothiazole-5-sulfonamide,
N-[2-(1H-indol-3-yl)ethyl]-2-methyl-1,3-benzothiazole-5-sulfonamide,
N-(4-iodobenzyl)-2-methyl-1,3-benzothiazole-5-sulfonamide,
N,N-diethyl-4-(2-{[(2-methyl-1,3-benzothiazol-5-yl)sulfonyl]amino}ethyl)benzamide,
2-methyl-N-[4-(trifluoromethoxy)benzyl]-1,3-benzothiazole-5-sulfonamide,
2-methyl-N-[(3-phenylisoxazol-5-yl)methyl]-1,3-benzothiazole-5-sulfonamide, and
2-methyl-N-[(2-phenyl-1,3-thiazol-4-yl)methyl]-1,3-benzothiazole-5-sulfonamide,
or salts, solvates or solvated salts thereof.

8. A compound according to claim 1, for use in therapy.

9. A compound according to claim 1, in treatment of VR1 mediated disorders.

10. A compound according to claim 9 for treatment of acute and chronic pain, acute and chronic neuropathic pain and acute and chronic inflammatory pain.

11. A compound according to claim 9 for treatment of respiratory diseases.

12. A method of treatment of VR1 mediated disorders and for treatment of acute and chronic pain, acute and chronic neuropathic pain and acute and chronic inflammatory pain, and respiratory diseases, comprising administering to a mammal, including man in need of such treatment, a therapeutically effective amount of the compound of formula I, according to any one of claim 1.

13. A pharmaceutical formulation comprising as active ingredient a therapeutically effective amount of the compound of formula I, according to claim 1, in association with one or more pharmaceutically acceptable diluents, excipients and/or inert carriers.

14. The pharmaceutical formulation according to claim 13, for use in the treatment of VR1 mediated disorders and for treatment of acute and chronic pain, acute and chronic neuropathic pain and acute and chronic inflammatory pain, and respiratory diseases.

15. A processes for the preparation of the compound according to claim 1, wherein R¹ to R⁴, m, n and p, are defined as in claim 1, comprising; 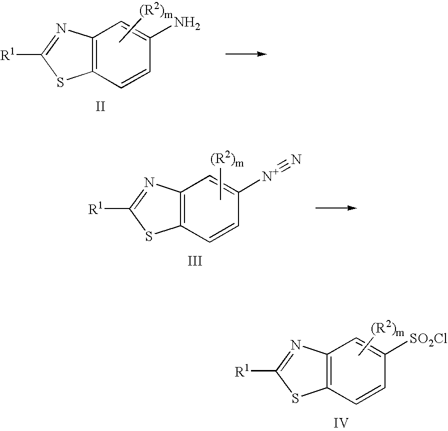
reaction of an aromatic amine of formula (II) with sodium nitrite in the presence of an acid to form a diazonium intermediate (III) which in turn is reacted in-situ with sulphur dioxide or sodium sulfite in the presence of copper chloride to form an aromatic sulfonyl chloride (IV), followed by 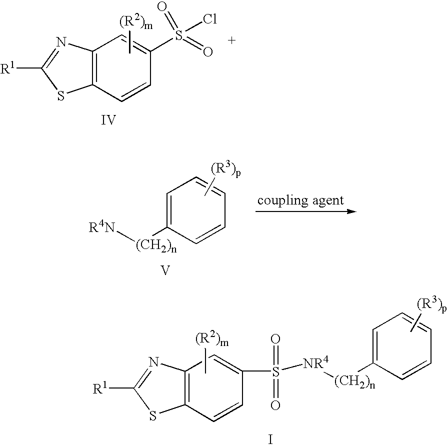
b) reaction of an aromatic sulfonyl chloride (IV) with a properly substituted amine (V) in the presence of a base.

16. The process for the preparation of the compound of formula I, wherein R¹ to R⁴, m, n and p, as defined in claim 1, comprising; 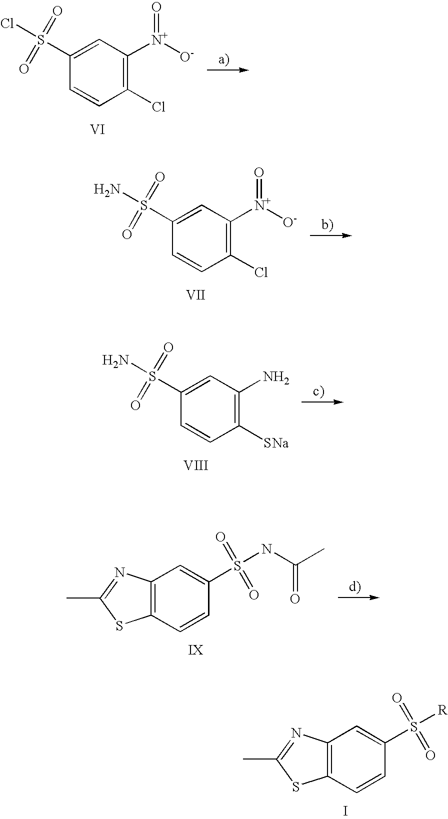
and wherein R is

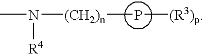

17. The compound N-[(2-methyl-1,3-benzothiazol-5-yl)sulfonyl]acetamide.

18. The compound allyl (5-amino-1,3-benzothiazol-2-yl)methyl carbonate.

19. The compound according to claim 17 used as an intermediate in the preparation of a compound of formula I.

20. The compound according to claim 18 used as an intermediate in the preparation of a compound of formula I.
